(19) United States
(12) Patent Application Publication
(10) Pub. No.: US 2016/0270411 A1
(43) Pub. Date: Sep. 22, 2016

(54) WHEAT BRAN SOLUBLE EXTRACT AS ANTI-BIOFILM AGENT

(71) Applicant: UNIVERSITAT AUTONOMA DE BARCELONA, Bellaterra (Cerdanyola del Valles) (ES)

(72) Inventors: Susana Maria MARTÍN ORÚE, Bellaterra (ES); Jose Francisco PÉREZ HERNÁNDEZ, Bellaterra (ES); Gemma GONZÁLEZ ORTÍZ, Bellaterra (ES)

(73) Assignee: UNIVERSITAT AUTONOMA DE BARCELONA, Bellaterra (ES)

(21) Appl. No.: 15/036,103

(22) PCT Filed: Nov. 14, 2014
(86) PCT No.: PCT/EP2014/074608

§ 371 Date: May 12, 2016

(30) Foreign Application Priority Data
Nov. 15, 2013 (EP) 13193093.5

(51) Int. Cl. A01N 65/44; A61K 38/56; A61K 38/47; A61K 36/899

(52) U.S. Cl. CPC A01N 65/44; A61K 36/899; A61K 38/56; A61K 38/47; C12Y 302/01014; C12Y 302/01002

(57) ABSTRACT

The present invention relates to products, compositions, methods and uses thereof which are useful in relation to the prevention and treatment of pathogen infections, preferably bacterial infections. More specifically, the invention relates to the use of wheat bran soluble extract as bacterial anti-biofilm agent.

### FIELD OF THE INVENTION

The invention relates to products, compositions, methods and uses thereof which are useful in relation to the prevention and treatment of pathogen infections, preferably bacterial infections. More specifically, the invention relates to the use of wheat bran soluble extract as bacterial anti-biofilm agent. It is interesting to note that the wheat bran soluble extract is also used to interfere the bacterial adhesion to epithelium agent.

### STATE OF THE ART

Bacterial biofilms are broadly defined as communities of bacterial cells encased in a protective extracellular matrix. These bacterial reservoirs are difficult to combat, leading to subsequent chronic infections that can persist despite the use of antibiotic treatments. Moreover, in some cases low doses of antibiotics can even enhance biofilm formation suggesting a natural defence mechanism of bacteria in avoiding the lethal effects of antibiotics.

The management of bacterial infections is becoming increasingly difficult due to the emergence and increasing prevalence of bacterial pathogens that are resistant to antibiotics. Due to this problematic, in veterinary practice, as well as in human medicine, alternatives to antibiotic therapies are increasingly required. As an example, mastitis is one of the most important diseases in dairy cattle being on the top among diseases related with biofilm production. Staphylococcus aureus is the prime etiological agent causing mastitis in bovines. Besides the production of many microbial surface components recognizing adhesive matrix molecules (MSCRAMMs) and mediating epithelial attachment, the formation of biofilms is also recognized as an important virulence factor in S. aureus. The general therapeutic approach towards mastitis is to use antibiotics to combat underlying infection. However, bacteria in biofilms are significantly less responsive to antibiotics and antimicrobial stressors than planktonic organisms of the same species, and S. aureus may resist many therapeutic regimes.

Consequently, several biological strategies for combating bacterial biofilms have been described in the state of the art. Some of these alternatives are: antibody therapy, synthetic peptide vaccine, disruption of biofilm extracellular polymeric substance (EPS) matrix, negating biofilm formation by disrupting iron metabolism and inhibiting or negating cell-cell signalling.

It is generally accepted that in a number of clinically relevant bacterial pathogens, most notably in S. aureus and P. aureuginosa, Quorum Sensing (QS) signalling plays an important role in the control of virulence factor expression as well as biofilm formation. In this sense, QS is an important regulatory mechanism of biofilm lifestyle in a variety of bacterial species. The QS involves the accumulation of signalling molecules in the surrounding environment which enables a single cell to sense the density of the number of bacteria and the signalling molecules, and therefore the population as a whole, can make a coordinated response. These cell-cell communication systems regulate various functions as diverse as motility, virulence, sporulation, antibiotic production, DNA exchange, and development of more complex multicellular structures such as biofilm. Therefore, targeting of QS signalling systems might offer a new strategy to combat bacterial infections. Approaches that interfere with proper microbial QS signalling are called “quorum quenching (QQ)”.

Moreover, the possibility that pathogens can be inhibited by naturally occurring anti-adhesive compounds is especially attractive and has captured significant attention. Milk source, plant-derived compounds, and microbial by-products have been the most important dietary products considered for this function. Vegetable products with anti-adhesion activity are attractive candidates as therapeutic agents, because they are generally abundant or can be engineered to become widely available. Consequently, natural sources may provide an alternative tool to antibiotics to fight against bacterial infections by interfering the bacterial adhesion, but also with virulence factors and mechanism of communication between bacteria as QS.

In recent years, the screening and evaluation of foods with QQ activity have gained increasing popularity. It is not strange that natural sources would provide a lot of QQ agents since they might have co-evolved to prevent bacterial encroachment and infection or to outcompete microbial counterparts in the environment. Despite the fact that a plethora of QQ agents have been discovered and characterized in vitro and in vivo, no candidate has reached clinical stage development yet. Therefore, looking for natural compounds with QQ properties to prevent biofilm formation and microbial adhesion may be a feasible alternative, thus reducing antimicrobial resistance.

The document WO 2012/0154242 A2 (UNIV HANNAM INST IND ACAD COOP) pertains to an antimicrobial composition and discloses a composition comprising wheat bran extract. However this document does not lie within the scope of this invention because the current invention claims anti-biofilm and bacterial anti-adhesive properties of the wheat bran extract, fraction and compounds as these terms are defined in the description.

The document WO 2011/081489 A2 (LEE ET AL) discloses the use of the wheat bran extract to treat allergic diseases which is not the aim of the current invention. This document is different from the invention in that wheat bran extract or 5-n-non-adcylresorcinol or 5-n-heneicosylresorcinol are used as inhibitors of the degranulations of fat cells, the formation of cytokine, and the like, and the composition is not used and/or claimed as anti-biofilm and bacterial anti-adhesive agent.

The document US 2013/102554 A1 (LEE ET AL) discloses a composition for treatment of obesity using a wheat bran extract. This document is not considered to lie within the scope of the current invention. This document is different from the invention in that the composition is not used and/or claimed as anti-biofilm and bacterial anti-adhesive agent.

Although the ability of a bacteria to attach a surface is a first step to initiate the development of a biofilm structure, it is not only the only requirement, actually after the initial binding and irreversible adhesion to the colonized surface, the bacteria proliferate and accumulate as multilayered cell cluster producing extracellular matrices composed of a mixture of materials such as polysaccharides, proteins or nucleic acids. After this initial phase, the microbial biofilm enter into a maturation phase in which characteristic channel-containing biofilm structures are formed and ultimately facilitate the last stage of biofilm development that is the detachment phase. This last stage has a fundamental role in the further dissemination of the biofilm. Therefore inhibiting the biofilm formation avoiding bacterial adhesion could imply the possibility of interfere in any of multiple steps even in their ability to produce the extracellular matrix or e interfere in any of the complex molecular determinants that could be involved in the different biofilm development phases.

In this sense, the present invention disclosed that the wheat bran (WB) extract from Tricicum aestivum has anti-biofilm activity against pathogenic bacteria and also it is able to interfere with bacterial QS. The data show that the WB extract exhibits anti-biofilm activity, inhibiting the biofilm formation and/or destroying the biofilm previously formed. The soluble extract of WB also showed a potential to interfere with the QS of the bacteria as it was demonstrated to contain certain lactanase activity able to reduce acyl-homoserine lactone (AHL) activity. Therefore, the present invention reveals two additional beneficial properties of WB extract, which may be related to the presence of defence compounds in the plant extract able to interfere with microbial biofilms and also QS systems. Moreover, soluble extract of WB also showed anti-adhesive properties of enteropathogenic bacteria to the intestinal mucus and to the intestinal epithelium. Specifically, in the present invention it is disclosed the active compounds responsible of the anti-biofilm and anti-adhesion activity comprised in the soluble extract of WB, showing that the active compounds are mostly retained in the >300-kDa fraction of the soluble extract.

It is important to note that wheat is by far, one of the most important cereals produced worldwide (FAO-Food and Agriculture Organization. 2013), and as a consequence WB is an easy by-product to find worldwide in contrast to other natural products that are not easily available (Quave C. L. et al. J. Ethnopharmacol. 2008; 118: 418-428). Wheat bran is rich in carbohydrates (60%), protein (12%), fat (0.5%), minerals (2%) and several bioactive compounds and vitamins (Mohsin-Javed, M. et al. African Journal of Microbiology Research. 2012; 6: 724-733). Therefore the soluble extract of WB becomes a natural product with bacterial anti-biofilm and anti-adhesion properties which it is not expected to generate residuals in the animal or the products, or to have an environmental impact like other alternatives as antibiotics or toxicities effects as it is not acting as a bactericidal substance. Moreover, the anti-biofilm activity of WB will theoretically improve the activity of other antimicrobial compounds, such as antibiotics. Also being a product able to inhibit the virulence of the pathogens but not their growth. Moreover, it would diminish considerably the risk of selection of antimicrobial resistances like antibiotics do.

### DESCRIPTION OF THE INVENTION

### Brief Description of the Invention

In a first aspect, the invention relates to wheat bran soluble extract for use as bacterial anti-biofilm agent. Therefore, the wheat bran soluble extract disclosed in the invention has also to be used to interfere the bacterial adhesion to epithelium agent.

In this sense, the wheat bran soluble extract, specifically the fraction >300-kDa of that soluble extract has anti-adhesive and anti-biofilm activity against pathogenic bacteria. The WB extract exhibits anti-biofilm activity, inhibiting the biofilm formation and/or destroying the biofilm previously formed. The soluble extract of WB also showed a potential to interfere with the QS of the bacteria as it was demonstrated to contain certain lactanase activity able to reduce acyl-homoserine lactone (AHL) activity. Moreover, as previously mentioned, the soluble extract of WB also shows anti-adhesive properties of enteropathogenic bacteria to the epitheliums, preferably to the intestinal mucus and to the intestinal epithelium.

It is important to note that the present invention disclosed the specific active compounds responsible of the anti-biofilm and anti-adhesion activity comprised in the soluble extract of WB, showing that these active compounds are mostly retained in the >300 kDa-fraction of said soluble extract. Among these active compounds stands out the presence of various protease inhibitors (PIs) of low molecular weight and a storage protein 7S globulin of wheat, the Globulin 3 of 66-kDa, which seem to be one of the most firmly attached WB proteins to the enterophatogenic bacteria.

In a second aspect, the invention relates to the protease inhibitors selected from any of the following: Serpin-Z2B, Class II chitinase, endogenous alpha-amylase/subtilisin inhibitor and alpha-amylase/trypsin inhibitor CM3 and/or Globulin 3 of wheat, for use as bacterial anti-biofilm agent and/or bacterial anti-quorum sensing signaling, and moreover in a preferred embodiments as bacterial anti-adhesive agent, preferably in the prevention and/or treatment of infections caused by pathogenic biofilm-forming bacteria. In a preferred embodiment, the pathogenic biofilm-forming bacteria are selected from: Staphylococcus; Streptococcus; Mannheimia; Pasteurella; Pseudomonas; Burkolderia; Hemophilus; Legionella; Fusobacterium; Corynebacterium; Escherichi, Salmonella; Listeria; Vibrio; Porphyromonas; Enterococcus; Neisseria; and Pseudomonas. In another preferred embodiment, the pathogenic biofilm-forming bacteria pertains to the species Staphylococcus aureus Streptococcus agalactiae, Mannheimia haemolytica, Pasteurella multocida, Pseudomonas aeruginosa, Burkolderia cepacia, Streptococcus neumoniae, Hemophilus influenza, Legionella neumophila Fusobacterium necrophorum, Corynebacterium pseudotuberculosis, Streptococcus spp., Porphyromonas gingivalis, Pseudomonas aeruginosa, Enterococcus faecalis, Neisseria gonorrhoeae, Escherichia coli, Salmonella enteritidis, and Pseudomonas aeruginosa.

In a more preferred embodiment, the protease inhibitors and/or the Globulin 3 are used in the prevention and/or treatment of mastitis, pneumonia, liver abscess, lymphadenitis, enteritis, dental caries, otitis, urinary infections, gonhorrea and wound infections. Alternatively, the wheat bran soluble extract, according to the present invention it is also characterized for use for the manufacturing of an antimicrobial agent for the treatment and/or prevention of mastitis, pneumonia, liver abscess, lymphadenitis, dental caries, otitis, urinary infections, gonhorrea enteritis and wound infections.

In still another aspect, the invention relates to a composition selected from: pharmaceutical, prebiotic or functional food compositions, comprising wheat bran soluble extract; or a protease inhibitor selected from: Serpin-Z2B, Class II chitinase, endogenous alpha-amylase/subtilisin inhibitor and alpha-amylase/trypsin inhibitor CM3; or Globulin 3, or combinations thereof, and excipients, inert ingredients or carriers; and, optionally, additional active principles or agents. The additional active principles or agents can be selected from: antibiotics and/or antiseptic solutions.

In still another aspect, the invention relates to a composition for bacterial control of facilities or devices selected from: industrial facilities, laboratories, including branches, farms, facilities, medical devices, water of liquid feed distribution system in farms, food industry, dairy industry, pipe networks for fluids, including air-conditioning systems comprising wheat bran soluble extract; or a protease inhibitor selected from Serpin-Z2B, Class II chitinase, endogenous alpha-amylase/subtilisin inhibitor and alpha-amylase/trypsin inhibitor CM3; or globulin 3 of wheat; and excipients, or carriers; and optionally additional active principle. The additional active principle can be selected from: antibiotics and/or antiseptic solutions.

The invention also deals with a method for the prevention and/or treatment of bacterial infections, selected from mastitis (Streptococcus agalactiae, Staphylococcus aureus), pneumonia (Mannheimia haemolytica, Pasteurella multocida, Pseudomonas aeruginosa, Burkolderia cepacia, Streptococcus neumoniae, Haemophilus spp., Legionella neumophila), liver abscess (Fusobacterium necrophorum), lymphadenitis (Corynebacterium pseudotuberculosis, Streptococcus spp.), enteritis (Escherichia coli, Salmonella spp., Listeria spp., Vibrio cholerae), dental caries (Streptococcus spp. Porphyromonas gingivalis), otitis (Pseudomonas aeruginosa, E. coli, Staphylococcus aureus, Haemophilus influenza) urinary infections (E. coli, Enterococcus faecalis), gonhorrea (Neisseria gonorrhoeae) and wound infections (Staphylococcus aureus, Pseudomonas aeruginosa), comprising the administration to a subject in need thereof of an effective dose of the wheat bran soluble extract, as defined in the present invention.

The invention further provides a method for the prevention and/or treatment of biofilm formation in an environment comprising the step of administering to the environment the wheat bran soluble extract, or the protease inhibitors selected from any of the following: Serpin-Z2B, Class II chitinase, endogenous alpha-amylase/subtilisin inhibitor and alpha-amylase/trypsin inhibitor CM3 and/or Globulin 3 of wheat or the compositions disclosed in the present invention. Advantageously the method comprises the step of administering to the environment a product according to the invention, which inhibits the biofilm formation and/or destroying the biofilm previously formed and the adhesion of pathogens to living epitheliums like intestine. Moreover, also interfere with the QS of the microorganisms.

The method of the invention may be used to minimise and, preferably, prevent the formation of biofilms in a variety of environments including, but not limited to, household, workplace, laboratory, industrial environment, aquatic environment (e.g.pipeline systems), medical devices including indwelling devices such as defined herein, dental devices or dental implants, animal body for example human body.

### DESCRIPTION OF THE FIGURES

FIG. 1. Dose-response of the biofilm formation (%) in the biofilm inhibition assay using wheat bran (WB) extract and the fractions obtained by molecular weight. The tested samples were the WB extract, the >300-kDa fraction, the <300>100-kDa fraction and the <100-kDa fraction. PBS was included as a positive control to which compares each treatment. Data are results from two independent assays by duplicate. Bars represent standard error of the mean. For each concentration, the asterisks show significant differences regarding to PBS: *P<0.05; **P<0.01; ***P<0.001.

FIG. 2. Dose-response of the biofilm formation (%) in the biofilm destruction assay using wheat bran extract and the fractions obtained by molecular weight. The tested samples were the WB extract, the >300-kDa fraction, the <300>100-kDa fraction and the <100 kDa-fraction. PBS was included as a positive control to which compares each treatment. Data results from two independent assays by duplicate. Bars represent standard error of the mean. For each concentration, the asterisks show significant differences regarding to PBS: *P<0.05; **P<0.01; ***P<0.001.

FIG. 3. Biofilm formation (%) in both biofilm inhibition (A) and biofilm destruction (B) assays using WB-extract, the >300 kDa fraction and the eight obtained size exclusion chromatography fractions. PBS was included as a negative control and WB extract and the >300 kDa fractions were tested at 0.5%. Data results from two independent assays by duplicate. Data is represented as mean±standard error of the mean. Bars with unlike letters were significantly different (P<0.05).

FIG. 4. Relative response to the natural acyl-homoserine lactone (AHL) present in the positive control sample (%). The Relative Fluorescence Units (RFU) of samples were measured after the incubation of 900 μl of each sample with 100 μl of AHL for 24 h at 37° C. in the presence of a biosensor E. coli strain after 6 h of incubation. The values of samples were turned by giving the maximal percentage (100%) to the positive control sample. LB: luria broth (without added AHL); DS: AHL-degrader control strain (Pseudomonas fluorescens P3/pME6863); NDS: AHL non-degrader strain (Pseudomonas fluorescens P3/pME6000); Wheat bran extract equivalent to a 1:100 (w/v) suspension; >300-kDa fraction; <300 kDa-fraction. Bars represent standard error of the mean. Treatments with unlike letters were significantly different (P<0.05). Results were obtained from the average of three replicates in three independent assays.

FIG. 5. The adhesive (A) and anti-adhesive (B) ability of fractions were evaluated using the enterotoxigenic E. coli (ETEC) K88.

A) Number of bacteria (log CFU per well) attached to wells coated with the different molecular weight (MW) fractions obtained from wheat bran (WB) in the in vitro adhesion test (AT). The higher the log CFU counts than PBS, the higher adhesive ability. The samples tested were the WB extract (14 mg/ml), the >300 kDa fraction (2.7 mg/ml) and the <300 kDa fraction (17 mg/ml).
B) Number of bacteria (log CFU per well) that attached to IPEC-J2 cells after being co-incubated for 30 min with the different fractions obtained from WB. The lower the log CFU counts than PBS, the higher the blocking-adhesion ability. Different letters mean significant differences (P<0.05) between fractions. Data result from the experiments performed in triplicate in two independent assays. Error bars represent the standard error of the mean.

FIG. 6. Dose-response relationships of the ability of WB-extract (A) and the >300 kDa fraction (B) to block the attachment of enterotoxigenic E. coli K88 in the IPEC-J2 cell-line. Log CFU: number of bacteria attached to the intestinal cells that were not blocked when compared to PBS. The lower the log CFU counts, the higher the blocking-adhesion ability. Linear, quadratic and cubic contrasts were performed to analyse the dose-response. Data result from the experiments performed in triplicate in two independent assays. Error bars represent the standard error of the mean.

FIG. 7. Testing the recognition of enterotoxigenic E. coli (ETEC) K88 by the obtained size exclusion chromatography (SEC) fractions.

A) SEC of the >300 kDa fraction. Distribution of the 8 fractions obtained.
B) Number of bacteria (log CFU per well) attached to wells coated with the different fractions in the in vitro adhesion test (AT). The higher the log CFU counts than PBS, the higher adhesive ability.
C) Number of bacteria (log CFU per well) that attached to IPEC-J2 cells in the blocking test (BT). The lower the log CFU counts than PBS, the higher the blocking-adhesion ability.
In B and C figures, different letters mean significant differences (P<0.05) between fractions. Data results from two independent assays by triplicate. Error bars represent the standard error of the mean.
D) Dot blot analysis with purified fimbriae to immobilised WB-extract, >300 kDa and <300 kDa fractions, CGMP, BSA, fetuin and the eight fractions (F1 to F8). (i) Binding of purified K88ac fimbriae of ETEC strain FV12408. (ii) Binding of purified K88ac fimbriae of ETEC strain 5/95.
E) One dimension SDS-PAGE of the eight fractions obtained by SEC.

FIG. 8. Testing the recognition of enterotoxigenic E. coli K88 by the obtained size exclusion chromatography (SEC) fractions treated with acetonitrile.

A) SEC of the >300 kDa fraction treated with solvent. Representation of the 6 fractions obtained.
B) Number of bacteria (log CFU per well) attached to wells coated with the different fractions in the in vitro adhesion test (AT). The higher the log CFU counts than PBS, the higher adhesive ability.
C) Number of bacteria (log CFU per well) that attached to IPEC-J2 cells in the blocking test (BT). The lower the log CFU counts than PBS, the higher the blocking-adhesion ability.
In C and D figures, different letters mean significant differences (P<0.05) between fractions. Data result from two independent assays by triplicate. Error bars represent the standard error of the mean.
D) Dot blot analysis with purified fimbriae to immobilised WB-extract, >300 kDa, <300 kDa, CGMP, BSA, fetuin and the six fractions (FA1 to FA6). (i) Binding of purified K88ac fimbriae of ETEC strain FV12408. (ii) Binding of purified K88ac fimbriae of ETEC strain 5/95.
E) One dimension SDS-PAGE of the six fractions obtained by SEC treated with solvent. From B1 to B6 are indicated the excised bands which were identified by mass spectrometry.

FIG. 9. Isolation of wheat bran proteins attaching enterotoxigenic E. coli K88. ETEC K88 cells were incubated with PBS (1) and wheat bran extract (2). The proteins obtained after the shaving process were separated in 1D (A) and 2D (B) gels. The spots with a fold>2 are labelled as B7, B8 and B9.

### DETAILED DESCRIPTION OF THE INVENTION

All publications and patent applications herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

The authors of the present invention have observed that the wheat bran soluble extract from Tricicum aestivum shows special properties as anti-biofilm activity against pathogenic bacteria and also it is able to interfere with bacterial QS. Moreover, the wheat bran soluble extract also shows anti-adhesive properties to the intestinal epithelium. The data show that the WB extract exhibits anti-biofilm activity, inhibiting the biofilm formation and/or destroying the biofilm previously formed. The soluble extract of WB also showed a potential to interfere with the QS of the bacteria as it was demonstrated to contain certain lactanase activity able to reduce acyl-homoserine lactone (AHL) activity. Therefore, the present invention reveals that the soluble extract of WB also showed anti-adhesive properties of enteropathogenic bacteria to the intestinal mucus and to the intestinal epithelium. Specifically, in the present invention it is disclosed the active compounds responsible of the anti-biofilm and anti-adhesion activity comprised in the soluble extract of WB, showing that the active compounds are mostly retained in the >300-kDa fraction of said soluble extract.

### DEFINITIONS

As used herein the term “wheat bran” refers to the specific portion of the wheat grain obtained as by-product after wheat the milling process as shown in the present invention. It mostly consists of the combined aleurone and pericarp of the seed and some rest of endosperm. More specifically, the term “wheat bran soluble extract” refers to the soluble compounds obtained after the suspension of a variable percentage of WB in distilled water (ranging from 1-10% w/v), by the method discloses in the present invention. The supernatant obtained (or the dry residue obtained after freeze drying this supernatant) constitute what is defined as “wheat bran soluble extract”

As used herein, the term “biofilm” refers to a population of microorganisms (bacteria, fungi, and/or protozoa, with associated bacteriophages and other viruses) that are concentrated at an interface (usually solid/liquid) and typically surrounded by an extracellular polymeric slime matrix. Biofilms may form on a wide variety of surfaces, including living tissues, indwelling medical devices, industrial or potable water system piping, or natural aquatic systems.

As used herein, the term “anti-biofilm agent” refers to an agent that is capable of destroying or inhibiting the growth of a microbial biofilm. The anti-biofilm agent may be capable of disrupting the structure of the biofilm, for example the extracellular mucous matrix.

As used herein, the term, “anti-quorum sensing signalling agent” refers to an agent that is capable of interfere with proper microbial QS signalling. Furthermore, this agent is usually called “quorum quenching”.

The term “anti-adhesive agent” is intended to include any agent capable of inhibiting cell adhesion, proteins and organisms e.g. microbes thereby preventing the formation of a bacterial colony, in particular bacterial biofilm formation. In particular, the anti-adhesive agent may prevent the adhesion (without killing bacteria) to a static or live surface of all cell types encountered in microbial biofilms in particular free living microbes.

As used herein, the terms “inhibit”, “inhibiting” and “inhibition” refer to stopping, preventing, reducing or eliminating the biofilm formation, the bacterial anti-quorum sensing signalling and the adhesion to the epithelium.

As used herein, the term “subject” or “patients” refers to any animal, human or non-human, including, but not limited to, humans, non-human primates, rodents, avians and the like, which is to be the recipient of a particular treatment. Typically, the terms “subject” or “patient” are used interchangeably across to the present invention. As used herein, the term “non-human animals” refers to all non-human animals including, but are not limited to, vertebrates such as rodents, non-human primates, ovines, bovines, ruminants, lagomorphs, porcines, caprines, equines, canines, felines, avians, etc.

As used herein, the term “active ingredient or active principle or active agent” refers to compounds, drugs, agents, substances, or materials which are known or are otherwise effective in providing a therapeutic benefit that can act locally and/or systemically in the body or in the environment. The term “active agent” includes agents that can be administered to a subject or surfaces for the treatment (e.g., therapeutic agent) or prevention (e.g., prophylactic agent) of a disease or disorder or infection. The “active ingredient” includes those known or otherwise effective pharmaceutical compounds or materials. Reference to a specific active agent shall include where appropriate the active agent and its pharmaceutically acceptable salts.

The term “treatment”, as used herein, refers to the act of reversing, alleviating, or inhibiting the progression of the disorder or condition to which such term applies, or one or more symptoms of such disorders or condition.

The term “prevention”, as used herein, refers to the act of keeping from happening, existing, or alternatively delaying the onset or recurrence of a disease, disorder, or condition to which such term applies, or of one or more symptoms associated with a disease, disorder, or condition.

The term “prebiotics”, as used herein, refers to the use of substances that are added to foods, chewing gum, dietary supplements or others, which exert an effect on the composition of the host microbiota, characterised in that hindering the establishment of pathogenic bacteria, i.e., inhibit the adhesion to the epithelium and the biofilm formation of pathogen bacteria and also it is able to interfere with bacterial QS.

The term “functional food” as used herein, refers to those foods that are prepared not only for their nutritional characteristics, but also to fulfil a specific function, such as improving health or reducing the risk of contracting diseases. To this end, biologically active compounds, such as minerals, vitamins, fatty acids, bacteria with beneficial effects, dietary fibre and antioxidants, etc., are added thereto.

In a first object, the present invention refers to the wheat bran soluble extract for use as bacterial anti-biofilm agent. Alternatively, the invention also discloses the use of the wheat bran soluble extract in the manufacturing of bacterial anti-biofilm agent.

In a preferred embodiment, the wheat bran soluble extract for use according to the present invention also is used as bacterial anti-adhesive agent to the intestinal epithelium.

In a preferred embodiment, the use of wheat bran soluble extract it is characterized in that the soluble extract consist in a fraction >300-kDa. This fraction is obtained, according to the present invention, after centrifugation of the wheat bran soluble extract using appropriate devices provided with a membrane with a cut-off size of 300,000-Da.

In another preferred embodiment, the use of wheat bran soluble extract, according to the present invention, it is characterized in that said wheat bran soluble extract comprises at least one protease inhibitors selected from: Serpin-Z2B, Class II chitinase, endogenous alpha-amylase/subtilisin inhibitor and alpha-amylase/trypsin inhibitor CM3.

In another preferred embodiment, the use of wheat bran soluble extract, according to the present invention, it is characterized in that said wheat bran soluble extract comprises the Globulin 3 of wheat.

In another embodiment, the use of wheat bran soluble extract, according to the present invention, it is characterized in that the bacteria treated are a biofilm forming bacteria.

In another preferred embodiment, the wheat bran soluble extract is for use, according to the present invention, in biofilm forming bacteria that pertain to the genus Staphylococcus; Streptococcus; Mannheimia; Pasteurella; Pseudomonas; Burkolderia; Hemophilus; Legionella; Fusobacterium; Corynebacterium; Escherichi; Salmonella; Listeria; Vibrio; Porphyromonas; Enterococcus; Neisseria; and Pseudomonas.

In another preferred embodiment, the use of wheat bran soluble extract, according to the present invention it is characterized in that the bacteria pertains to the species Staphylococcus aureus, Streptococcus agalactiae, Mannheimia haemolytica, Pasteurella multocida, Pseudomonas aeruginosa, Burkolderia cepacia, Streptococcus neumoniae, Hemophilus influenza, Legionella neumophila, Fusobacterium necrophorum, Corynebacterium pseudotuberculosis, Streptococcus spp., Porphyromonas gingivalis, Pseudomonas aeruginosa, Enterococcus faecalis, Neisseria gonorrhoeae, Escherichia coli, Salmonella enteritidis and Pseudomonas aeruginosa.

In another preferred embodiment, the wheat bran soluble extract, according to the present invention, it is characterized for use in the prevention and/or treatment of diseases induced by biofilm-forming bacteria. In a more preferred embodiment, the diseases induced by biofilm-forming bacteria are selected from: mastitis, pneumonia, liver abscess, lymphadenitis, enteritis, dental caries, otitis, urinary infections, gonhorrea and wound infections. Alternatively, the wheat bran soluble extract, according to the present invention it is also characterized for use for the manufacturing of bacterial anti-biofilm agent for the treatment and/or prevention of diseases induced by biofilm-forming bacteria. In a more preferred embodiment, the diseases induced by biofilm-forming bacteria are selected from: mastitis, pneumonia, liver abscess, lymphadenitis, dental caries, otitis, urinary infections, gonhorrea, enteritis and wound infections.

In another preferred embodiment, the wheat bran soluble extract for use according to the present invention it is characterized in that are used in combination, simultaneously, sequentially or separately, with at least one additional active principle or agent. In a more preferred embodiment, the active principle or agent is selected from: antibiotics and/or antiseptic solutions.

Other object of the present invention refers to the protease inhibitor selected from Serpin-Z2B, Class II chitinase, endogenous alpha-amylase/subtilisin inhibitor and alpha-amylase/trypsin inhibitor CM3 for use as bacterial anti-biofilm agent and/or alternatively, the use of protease inhibitor selected from Serpin-Z2B, Class II chitinase, endogenous alpha-amylase/subtilisin inhibitor and alpha-amylase/trypsin inhibitor CM3, in the manufacturing of a bacterial anti-biofilm agent.

In a preferred embodiment, the protease inhibitors disclosed in the present invention are used in the prevention and/or treatment of bacterial diseases selected from: mastitis, pneumonia, liver abscess, lymphadenitis, enteritis dental caries, otitis, urinary infections, gonhorrea and wound infections. Alternatively, the present invention also discloses the use of the protease inhibitors disclosed in the present invention in the manufacturing of anti-microbial agents for the prevention and/or treatment of bacterial diseases selected from: mastitis, pneumonia, liver abscess, lymphadenitis, enteritis, dental caries, otitis, urinary infections, gonhorrea and wound infections.

Other object of the present invention refers to the Globulin 3 of wheat for use as bacterial anti-biofilm agent and/or alternatively, the use of the Globulin 3 of wheat, in the manufacturing of a bacterial anti-biofilm agent.

In a preferred embodiment, the Globulin 3 of wheat is used in the prevention and/or treatment of bacterial diseases selected from: mastitis, pneumonia, liver abscess, lymphadenitis, enteritis dental caries, otitis, urinary infections, gonhorrea and wound infections. Alternatively, the present invention also discloses the use of the Globulin 3 of wheat in the manufacturing of anti-microbial agents for the prevention and/or treatment of bacterial diseases selected from: mastitis, pneumonia, liver abscess, lymphadenitis, enteritis, dental caries, otitis, urinary infections, gonhorrea and wound infections.

Other object of the present invention refers to a composition selected from: pharmaceutical, prebiotic or functional food compositions, comprising wheat bran soluble extract; or a protease inhibitor selected from Serpin-Z2B, Class II chitinase, endogenous alpha-amylase/subtilisin inhibitor and alpha-amylase/trypsin inhibitor CM3; or Globulin 3 of wheat; and excipients, inert ingredients or carriers; and optionally additional active principle or agent. In a more preferred embodiment, the active principle or agent is selected from: antibiotics and/or antiseptic solutions.

Other object of the present invention refers to a composition for bacterial control of facilities or devices selected from: industrial facilities, laboratories, including branchs, farms, facilities, medical devices, water of liquid feed distribution system in farms, food industry, dairy industry, pipe networks for fluids, including air-conditioning systems comprising wheat bran soluble extract; or a protease inhibitor selected from Serpin-Z2B, Class II chitinase, endogenous alpha-amylase/subtilisin inhibitor and alpha-amylase/trypsin inhibitor CM3; or Globulin 3 of wheat; and excipients, or carriers; and optionally additional active principles or agents. In a more preferred embodiment, the active principles or agents is selected from: antibiotics and/or antiseptic solutions.

Any suitable carrier, excipient or inert ingredients can be used in the present compositions (See e.g., Remington: The Science and Practice of Pharmacy, Alfonso R. Gennaro (Editor) Mack Publishing Company, April 1997).

In yet another aspect, the invention relates to a method for the treatment and/or prevention or bacterial diseases or infections selected from: mastitis, pneumonia, liver abscess, lymphadenitis, enteritis, dental caries, otitis, urinary infections, gonhorrea and wound infections, comprising the administration to a subject in need thereof of wheat bran soluble extract according to the present invention, or the protease inhibitors according to the present invention, or the Globulin 3 according to the present invention or an compositions according to the present invention.

The invention further provides a method for the prevention and/or treatment of biofilm formation in an environment comprising the step of administering to the environment the wheat bran soluble extract, or the protease inhibitors selected from any of the following: Serpin-Z2B, Class II chitinase, endogenous alpha-amylase/subtilisin inhibitor and alpha-amylase/trypsin inhibitor CM3 and/or Globulin 3 of wheat or the compositions disclosed in the present invention. Advantageously the method comprises the step of administering to the environment a product according to the invention. A substance or compound which inhibits the biofilm formation and/or destroying the biofilm previously formed and the adhesion of pathogens to living epitheliums like the intestine. Moreover, also interfere with the QS of the microorganisms.

The method of the invention may be used to minimise and, preferably, prevent the formation of biofilms in a variety of environments including, but not limited to, household, workplace, laboratory, industrial environment, aquatic environment (e.g.pipeline systems), industrial facilities, laboratories, including branchs, farms, facilities, medical devices, water of liquid feed distribution system in farms, food industry, dairy industry, pipe networks for fluids, including air-conditioning systems medical devices including indwelling devices such as defined herein, dental devices or dental implants, animal body including human body.

The dose of wheat bran soluble or the protease inhibitors, or the Globulin 3 or the compositions and the time of administration of such compositions will be within the purview of the skilled artisan having benefit of teachings present in the state of the art. In fact, the inventors contemplate that the administration of therapeutically-effective amounts of the wheat bran soluble extract of the invention may be achieved by a single administration. Alternatively, in some circumstances, it may be desirable to provide multiple, or successive administrations of the wheat bran soluble extract or the compositions of the invention, either over a relatively short, or a relatively prolonged period of time, as may be determined by the medical or veterinarian practitioner overseeing the administration of such compositions.

The term “therapeutically effective amount” or “therapeutically effective dose” refers to the amount of the compound, wheat bran soluble extract, protease inhibitors or Globulin 3, or compositions, etc as disclosed in the present invention, that will elicit the biological or medical response of a tissue, system, animal or human that is being sought by the researcher, veterinarian, medical doctor or other clinician. These terms include that amount of a compound, wheat ban soluble extract, protease inhibitors, Globulin 3, compositions, etc., as disclosed in the present invention that, when administered, is sufficient to prevent the development of, or alleviate to some extent, one or more of the symptoms of the condition or disorder or environments being treated. The therapeutically effective amount will differ depending on the wheat bran soluble extract, protease inhibitors, or Globulin 3, or compositions, etc as disclosed in the present invention, the disease and its severity and the age, weight, etc., of the mammal to be treated.

The present invention finds use in environments and in veterinary and medical applications. Suitable subjects include both avians and mammals. The term “avian” as used herein includes, but is not limited to, chickens, ducks, geese, quail, turkeys and pheasants. The term “mammal” as used herein includes, but is not limited to, humans, bovines, ovines, caprines, equines, felines, canines, lagomorphs, etc. Human subjects are the most preferred. Human subjects include infant, juvenile and adult subjects.

Administration of the wheat bran soluble extract and the compositions of the present invention to a mammal, including human subject or an animal in need thereof could be carried out by different routes of administration known in the art. For example, routes of administration include oral, intranasal, intraocular injections, intravenous, subcutaneous, sublingual, topical, or by means of a transdermal patch. Injectable compositions can be prepared in conventional forms, either as liquid solutions or suspensions, solid forms suitable for solution or suspension in liquid prior to injection, or as emulsions.

Dosages of the wheat bran soluble extract will depend upon the mode of administration, the individual subject's condition and can be determined in a routine manner. Exemplary doses for the soluble extract achieving therapeutic or anti-biofilm effect in environments effects would be those obtained from suspensions between 0.1 and 10% wheat bran in water (w/v), being preferred a suspensions between 0.1-3%, being more preferred a suspensions between 0.1-1%.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

The invention is hereby explained by the following examples which are to be construed as merely illustrative and not limitative of the scope of the invention.

### Materials and Methods.

### Bacterial Strains and Media

The S. aureus strain BMA/FR/0.32/0074 was gently provided by Prof. Dr. J. Fink-Gremmels and was isolated from a mastitis episode in cows in the Netherlands. This strain is characterized by high slime production. One colony from a blood agar culture was used to inoculate 5 mL of tryptone soy broth (TSB)+0.25% glucose. The culture was incubated overnight at 37° C. with gentle shaking. This bacterial culture was used for the biofilm inhibition and the biofilm destruction assays.

Yersininia enterocolitica 057 was used to produce natural acyl-homoserine lactone (AHL). The strain, which was satisfactorily tested to produce AHL (Medina-Martinez, M. S. et al. J. Appl. Microbiol. 2007; 102: 1150-1158), was provided by the laboratory of Food Microbiology and Food Preservation (Ghent University, Ghent; Belgium). The sterile supernatant of a 24 h culture of Y. enterocolitica grown in Luria broth (LB), thus containing the natural AHL, was recovered after centrifugation (6,000×g, 5 min, room temperature) and filter sterilized (0.22 μm filters, Supor Acrodisc, Pall Life Science).

Two recombinant Pseudomonas fluorescens strains, P3/pME6863 and the derivate P3/pME6000 were used as degrader and non-degrader reference strains, respectively. Both strains were grown overnight at 30° C. in LB. Before use, the optical densities of the cultures were adjusted with sterile LB to an optical density of 0.5 at a wavelength of 600 nm.

Escherichia coli JB523, containing the plasmid pJBA130, responsible for the production of a green fluorescent protein, was used as an AHL biosensor.

An enterotoxigenic E. coli (ETEC) K88 strain isolated from a colibacillosis outbreak in Spain (Blanco, M. et al. J. Clin. Microbiol. 1997; 35, 2958-2963) (strain reference n°: FV12408), serotype (O149:K91:H10 [K-88]/LT-I/STb) that was generously provided by the E. coli Reference Laboratory, Veterinary Faculty of Santiago de Compostela University (Lugo) was used. ETEC K88 was cultured in unshaken Luria broth at 37° C. Bacteria were serially cultured every 24 h, at least three times. These bacterial cells were collected by centrifugation of 15 mL of an overnight culture (1,700×g, 5 min, 20° C.). Supernatants were removed and PBS buffer was added to the cell pellet to achieve an optical density (OD) of 1 (650 nm) for the bacterial suspension that was used in the adhesion test (approximately log 8.5-9 CFU [colony forming units]/mL). For the blocking test, the bacterial suspension was serially diluted to 6.5-7 log CFU/mL. To isolate K88 fimbriae the wild-type ETEC strains FV12408 and 5/95 (O149, K88ac, LT+STb+; Joensuu, J. J. et al. Transgenic Res. 2006; 15, 359-373) were cultured overnight at 37° C. in tryptone soy broth (TSB, 100 mL) with mild shaking (50 rpm) or on Luria agar (tetracycline 12.5 μg/mL), respectively. These bacteria were collected and suspended into PBS. The bacterial suspensions were vortexed for 2 to 4 minutes to detach fimbria) filaments from bacterial surfaces (Westerlund-Wikstrom, B. et al. Protein Eng. 1997; 10, 1319-1326). Next, the bacteria were pelleted and bacteria-free supernatants were analysed in 15% SDS-PAGE gels. After Coomassie staining the protein concentrations of fimbriae were determined densitometrically by Tina (v2.0) image analysis program using bovine serum albumin (BSA, Sigma) as protein concentration standards in the gels.

### Wheat Bran (WB) Extraction and Fractioning.

The WB used in the study comes from a Spanish local mill (Moreto, Mollet del Vallès, Barcelona). First, the WB was finely ground in an analytical grinder and then, was suspended in demineralized (DEMI) water to a solid-to-liquid ratio of 1:10 (w/v). Subsequently, the suspension was sonicated (J.P. Selecta S.A) three times for 30 s each and then centrifuged (460×g, 5 min, 20° C.). The supernatant extracted was divided into three aliquots. One aliquot was used just as it was to perform the heat treatment and the carbohydrate digestion. The second aliquot was freeze dried and stored at room temperature (RT), and finally the third aliquot was immediately fractionated by molecular weight.

Heat treatment. Wheat bran extract was heated during 30 min at 90° C. Sample was cooled and stored at −20° C. until use. The activity of the heated product was tested in the in vitro adhesion test.

Carbohydrate digestion. O-glycosidase (P0733S, 40,000,000 U/ml, New England BioLabs Inc.) combined with Neuraminidase (N2876, 1,000 mU/ml, Sigma) was used to catalyse the hydrolysis of O-linked saccharides and N-acetyl-neuraminic acid from glycoproteins and oligosaccharides maximizing the disappearance of sugars attached to proteins in the soluble extract of WB. The reaction was performed by mixing 2 mL of the soluble extract of WB 10% with 290 μL 10×G7 Reaction Buffer, 10 μL O-glycosidase (180 Units) and 25 μL Neuraminidase (11 Units). Samples were incubated at 37° C. for 1.5 h with gentle shaking. A negative control was also included, which consisted of the same volume of the sample mixed with DEMI water, instead of the reaction buffer and enzymes. To stop the enzymatic reaction, the digested samples were heated at 60° C. for 20 min. Samples were stored at −20° C.

Fractionation by molecular weight (MW). One aliquot of the soluble extract of WB was used to fractionate it by molecular weight using Vivaspin® 6 centrifugal concentrators (Sartorious) with a cut-off size of 300,000-Da (300-kDa) and 100,000 Da (100-kDa). The upper part of the tube was filled with the soluble extract and centrifuged (3,000×g, 3.5 h, 4° C.). After centrifugation, the upper part was adjusted with DEMI water to the same volume retrieved in the bottom container to achieve the same sample volume concentration. The <300-kDa fraction was divided in two aliquots, one of them was stored and the other one was submitted again to a 100,000-Da cut-off size filter, following the same centrifuging protocol and the same filling conditions. Finally, the following fractions were obtained: >300-kDa, <300-KDa, <300>100-kDa and <100-kDa. These fractions were stored at −20° C. until used.

Size exclusion chromatography (SEC). To further fractionate the >300-kDa fraction, size exclusion chromatography was performed on an AKTA Purifier System (GE Healthcare) using a High Load Superdex® 200 26/60 column (GE Healthcare). Thirty-five milligrams of the lyophilised >300-kDa fraction were re-suspended in 13 mL of PBS 0.25× and filtered through 0.22 μm filter units. The sample was injected into the column and eluted in a 1.5 column volume (CV) using an isocratic gradient of PBS 0.25× at a flow rate of 2.5 mL/min. The absorbance of the eluted fractions was monitored at 214 and 280 nm. Fractions were pooled in eight major fractions and were freeze-dried. They were resuspended at ¼ of their initial volume with DEMI water, so that the final buffer concentration was PBS 1×. The aliquots were stored at −20° C. until use. In order to disrupt protein aggregations, the same separation was repeated using acetonitrile (ACN) 20% in PBS 0.25× as chromatographic buffer. The fractions were pooled in six major fractions and after lyophilisation, fractions were resuspended at ¼ of the initial volume with DEMI water and kept at −20° C. until their use.

### Minimal Inhibitory Concentration (MIC) Assay.

A minimal inhibitory concentration (MIC) assay was performed for WB soluble extract to discard any possible antimicrobial effect on S. aureus. The MIC was determined in 96-well microtiter plates. Briefly, in a sterile flat bottom plate (Corning® Costar® Ref. n° 3599), a stock sample of WB-soluble extract was diluted (1:2) from 1% to 0.016% in TSB+0.25% glucose (final concentration 100 μL) by duplicate from rows A to G. Columns 3, 6, 9 and 12 were filled with additional 100 μL TSB+0.25% glucose medium. One-hundred microliters of the overnight cultured bacteria suspension (1:50 diluted) was added to the other columns. Row H served as a control well, where instead of sample, medium was added. Plates were covered with a breathseal and the lid and kept at 37° C. under static conditions overnight. Turbidity was measured by reading the optical density (OD) at 630 nm in a spectrophotometer reader (Biotek μQuant spectrophotometer). The readings were taken in two independent assays and in duplicate per trial.

### Biofilm Inhibition Assay.

A biofilm inhibition assay was employed to test the effect of WB extract, and the different fractions thereof, on inhibition of biofilm formation by S. aureus. This protocol is based according to Hensen's methodology (Melchior, M. B. et al. J. Vet. Med. B Infect. Dis. Vet. Public Health. 2006; 53: 326-332.) to stain the biofilm formed with safranin. Assays were performed in sterile round-bottom 96-well polystyrene plates (Corning@ Costar@ Ref. n° 3799). Each sample and dilution was tested in duplicate in two independent assays. A concentration of 1:100 of the samples soluble extracts were added in the first row wells. Samples were serially diluted (1:2) from 0.5% to 0.016% (100 μL of final sample volume). Then, the bacterial suspension was prepared by diluting 1:50 in TSB+0.25% glucose from the overnight culture and 100 μL were added to each well. Plates were closed with breathseal and the lid and were placed in a 37° C. incubator for 24 h. The supernatants were removed carefully, the wells were rinsed twice with DEMI water, and the biofilm was fixed with 200 μL 0.1M HCl for 1.5 h at room temperature (RT). The fixative was removed directly onto dry paper and 200 μL of safranin 0.1% were added to stain the biofilm during 1 h at RT. Safranin solution was removed and wells washed 4× with DEMI. Finally, 125 μL of 0.2M NaOH was added to all wells and incubated for 30 min at 57° C. to dissolve the safranin. Wells content were mixed, 100 μL were transferred to a non-sterile flat-bottom plate, and absorbance was measured in a spectrophotometer reader at 540 nm. The positive control (medium+bacteria) was considered to show good biofilm formation when the OD was above 0.2 (Biotek μQuant spectrophotometer). Positive controls and negative controls (medium+sample−without S. aureus) for sample background colour were included in each assay.

### Biofilm Destruction Assay.

The biofilm destruction assay was a modified version of the biofilm inhibition assay in which first the S. aureus are allowed to form the biofilm inside the wells for 48 h. Next, test samples are included to allow for destruction of the biofilm previously formed. This protocol is based on quantification of the remaining biofilm like in biofilm inhibition assay. Assays were performed in sterile round-bottom 96-well polystyrene plates (Corning® Costar® Ref. n° 3799). Each sample and dilution was tested in duplicate in two independent assays. Firstly, the bacterial suspension was prepared by diluting 1:200 in TSB+0.25% glucose from the overnight culture and 100 μL were added to each well. Plates were covered with breathseal film and the lid and were placed at 37° C. for 48 h. In a new sterile flat-bottom plate (Corning@ Costar@ Ref. n° 3599), samples concentration from 1:100 (w/v) were used to prepare serially dilutions (1:2) from 0.5% to 0.016%. The supernatants from the bacteria plates were removed carefully without disturbing the already formed biofilm. Then, 100 μL of the sample dilutions plate were transferred to the bacteria plate, and extra medium was added to a final volume of 200 μL/well. The plates were covered and incubated overnight at 37° C. The fixation with 0.1 HCL, the staining with 0.1% safranin and the dissolving with NaOH procedures were as described above for the biofilm inhibition assay. The remaining safranin content was measured in a spectrophotometer reader at 540 nm. The positive control (medium+S. aureus) was considered to show good biofilm formation when the optical density was above 0.2 (Biotek μQuant spectrophotometer). Positive controls and negative controls (medium+sample −without S. aureus) for sample background colour were included in each assay.

### Ability of Wheat Bran to Degrade Acil Homoserin Lactones (AHL).

The ability of WB extract and its fractions to degrade naturally produced AHL were evaluated in vitro according to Medina-Martinez et al. (Medina-Martinez, M. S. et al., J. Appl. Microbiol. 2007; 102: 1150-1158). Nine-hundred μL of WB-extract 1:100 (w/v) of the >300 kDa and the <300 kDa fractions were mixed with 100 μL of the naturally produced AHL. The recombinant P. fluorescens P3/pME6863 strain and the derivate P. fluorescens P3/pME6000 were used as degrader (DS) and non-degrader (NDS) control strains, respectively (Molina, L., et al. FEMS Microbiol. Ecol. 2003; 45: 71-81). The mixtures were incubated for 24 h at 30° C. with shaking (170 rpm). Afterwards, the mixtures were centrifuged (6,000×g, 5 min, RT) and filter sterilized. AHL detection was performed using an indirect fluorescence-based method according to Medina-Martinez et al. (Medina-Martinez, M. S. et al., J. Appl. Microbiol. 2007; 102: 1150-1158). The green fluorescence was detected by a μL plate fluorescence reader (FLx800®, Bio-Tek Instruments Inc., USA) where 100 μL of the biosensor strain was incubated with 50 μL of the suspensions for 6 h at 30° C. All samples were tested in triplicate in three independent assays. Positive (natural AHL produced) and negative (LB medium) controls were run in parallel. Data are presented as the relative response to AHL detection in positive controls (%).

### Adhesion Test (AT).

The ability of the different fractions to adhere to ETEC K88 was determined by using high-binding polystyrene μL plates in the in vitro adhesion test as described by Becker and co-workers (Becker, P. M. et al. J. Appl. Microbiol, 2007; 103, 2686-2696) and adapted by the inventors. Briefly, after different incubation and rinsing steps, the bacterial growth was monitored as OD at a wavelength of 650 nm at intervals of 10 min for 12 h (Spectramax® 384 Plus, Molecular Devices Corporation). All readings were performed in two independent assays and in triplicate per trial. The OD data were translated to colony forming units (CFU) by using the linear models (Gonzalez-Ortiz, G. et al. Br. J. Nutr, 2013; 19:1-10).

### Blocking Test (BT).

The cell line characteristics, the maintenance procedure and the blocking test protocol were followed according to González-Ortiz et al. (Gonzalez-Ortiz, G. et al. Vet. Microbiol. 2013; 19:1-10). Briefly, IPEC-J2 cells were subjected to a 24 h adaptation period in 96-well flat-bottom μL plates with a CO2-independent medium. Separately, a mixture (1:1) of each WB fraction (previously filter-sterilized) with the bacterial suspension was gently mixed in a 1.5 mL eppendorf, and 200 μL were immediately transferred to each well. The mixtures and cells were incubated for 30 min at 37° C., allowing non-blocked bacteria to attach to cells. Wells were washed once with PBS and, finally 200 μL of 002-independent medium was added. The bacterial growth monitoring procedures were managed as in the in vitro adhesion test protocol described above. Wheat bran extract and the >300 kDa and <300 kDa fractions obtained by molecular weight were subsequently submitted to a dose-response experiment. Each fraction was tested in triplicate in two independent assays. The OD data were translated to CFU by using the equations proposed by González-Ortiz et al. (Gonzalez-Ortiz, G. et al. Vet. Microbiol. 2013; 19:1-10).

### ETEC K88ac Fimbrial Binding to WB Fractions.

Binding of purified ETEC K88ac fimbriae to WB and the >300 kDa and <300 kDa fractions were tested in a dot blot assay (Virkola, R. et al. Infect. Immun. 1993; 61, 4480-4484) using ETEC K88ac fimbriae purified from the strain ETEC FV12408. Moreover, another purified K88ac fimbriae from the ETEC strain 5/95 previously used for similar purposes (Hermes R. G. et al. Comp. Immunol. Microbiol. Infect. Dis. 2012; 34: 479-488), was used to compare the binding ability of these fimbriae extracts. To confirm ETEC K88 fimbrial specific binding we included in the assay casein glycomacropeptide (CGMP) and fetuin (Sigma) as positive control targets as well as BSA, as a negative control (Hermes R. G. et al. Comp. Immunol. Microbiol. Infect. Dis. 2012; 34: 479-488). All target proteins from the WB extract, >300 kDa, <300 kDa, CGMP, BSA (5 μg/dot), and chromatographic fractions (2.5 μl/dot) as well as fetuin (2.5 μg/dot) were immobilized on nitrocellulose membranes. After blocking for 1 h at 37° C. in 2% (w/v) BSA/PBS, the membranes were washed three times with PBS containing 0.05% Tween 20® (PBS-Tween®) and incubated with the purified ETEC K88 fimbriae (20 μg/ml in 1% BSA/PBS-Tween®) overnight at 4° C. with gentle shaking. Dot blot membranes were washed three times with cold PBS-Tween® and incubated with anti-FaeG polyclonal serum (diluted 1:2000, Joensuu, J. J. et al. Transgenic Res. 2006; 15, 359-373) for 2 h at 4° C. After washing the membrane was incubated with alkaline phosphatase-conjugated anti-rabbit IgG (1:1000; DakoCytomation) for 2 h at 4° C., and the bound proteins were visualized by bromochloroindolylphosphatenitrobluetetrazolium (Sigma).

Rescuing WB Components that Bind to Fimbriae.

To identify the possible molecular compounds of WB interacting with fimbriae, an incubation of the bacteria with the whole extract of WB was performed. For the experiment we used the same bacterial conditions as described for the adhesion test.

Enterotoxigenic E. coli K88 and WB extract (3:1) were incubated for 30 min at 37° C. and incubation with PBS was also included as negative control. After the incubation, the bacteria cells were pelleted by centrifugation (1.700×g, 5 min, 20° C.) and washed by hand pipetting with 1 mL of PBS. The washing step was repeated four times. Finally, a treatment with Triton® X-100 at 1% for 10 min at RT eluted the WB-compounds that interacted with the bacteria. The supernatant was filtered through 0.22 μm pore-size filters and kept at −20° C.

Identification of the WB Components that Bind ETEC K88 Fimbriae.

One dimensional SDS-PAGE separation. Samples were treated with 2D Clean-Up Kit (GE Healthcare) and were resuspended in lysis buffer (8M urea, 2.5% Chaps, 2% ASB-14 and 40 mM DTT, pH 8.5). Samples were quantified using the Microplate BCA protein assay kit (Thermo Scientific). SDS-PAGE separation of the samples was performed using 12% acrylamide gels (BioRad). Fifteen micrograms of each sample were mixed with 6× loading buffer containing Tris HCl 0.35M pH 6.8, glicerol 30%, SDS 10%, 5% B-mercaptoethanol and were boiled for 5 min. Samples were separated at 15 mA for 30 min and 20 mA for 70 min. Proteins were stained with Instant Blue (Expedeon®) for 1 h at RT.

Two dimensional SDS-PAGE separation. The proteins rescued after the incubation between ETEC K88 and WB were separated by 2D SDS-PAGE. Samples were treated with the 2D Clean-Up Kit (GE Healthcare), resuspended in lysis buffer and quantified using the Microplate BCA protein assay kit (Thermo Scientific). Two-dimensional electrophoresis with immobilized pH gradients was carried out according to Bjellqvist, B. et al. (J. Biochem. Biophys. Methods, 1982; 6, 317-339). Briefly, first-dimension isoelectric focusing was performed on immobilized pH gradient strips (7 cm, pH 3-10) using an Ettan® IPGphor® System. Samples (15 μg) were applied by cup-loading, and after focusing at 14 kVh, strips were equilibrated for 15 min in 5 mL of equilibration solution (6 M urea, 100 mM Tris-HCl, pH 8, 30% v/v glycerol, 2% w/v SDS) with 10 mg/mL dithiothreitol (DTT) and then in 5 mL of equilibration solution with 22.5 mg/mL iodoacetamide) for 15 min, on a rocking platform. Second dimension SDS-PAGE was performed by laying the strips on 12.5% precast gels (BioRad). Gels were run at RT at constant amperage (15 mA/gel) until the bromophenol blue tracking front had run off the end of the gel.

Identification of proteins by mass spectrometry. The selected protein spots were excised from gels and digested in-gel. Before tryptic digestion, reduction and alkylation was performed by incubating samples with 10 mM DTT in 50 mM of ammonium bicarbonate for 30 min at RT, followed by alkylation with 25 mM iodoacetamide in 50 mM ammonium bicarbonate for 30 min at RT and protected from light. Protein digestion was accomplished using 25 ng of trypsin sequencing grade (Promega) for 3 h at 37° C. Peptides were eluted by centrifugation with 50 μL of ACN:H2O (1:1)+0.2% trifluoroacetic acid (TFA), evaporated using a speed-vacuum concentrator and resuspended in 5 μL of H₂O+0.1% TFA. For the mass spectrometry (MS) analysis all samples were prepared by mixing 0.5 μL sample with the same volume of a solution of •-cyano-4-hydroxycinnamic acid matrix (10 mg/mL in 30% ACN, 60% water+0.1% TFA) and were spotted onto a ground steel plate (Bruker Daltonics) and allowed to air-dry at RT. Matrix assisted laser desorption/ionization time-of-flight (MALDI-TOF) mass spectra were recorded in the positive ion mode on an Ultraflextreme mass spectrometer (Bruker Daltonics®). Ion acceleration was set to 25 kV. All mass spectra were externally calibrated using a standard peptide mixture. For peptide mass fingerprint analysis, the Mascot search engine (Matrix Science) was used with the following parameters: NCBInr database, 2 maximum missed trypsin cleavages, cysteine carbamidomethylation and methionine oxidation as variable modifications and 50 ppm tolerance. Positive identifications were accepted with scores over the significance threshold and P<0.05.

### Statistical Analysis.

Values from biofilm inhibition and biofilm destruction assays were analyzed with a generalized linear mixed model (GLMM) by using the GLIMMIX procedure of the statistical package of SAS 9.2 (SAS Inc.; Cary, N.C., USA). For both parameters the model was fitted to an inverse Gaussian distribution for the analysis. The relative response to AHL detection in positive controls was analyzed with the same procedure fitting the model to a negative binomial distribution.

The OD data from the adhesion test and the blocking test were processed by non-linear regression analysis using the non-linear P-NLIN (Gauss-Newton method) procedure (SAS 9.2, SAS Inc., Cary, N.C., USA) following the equations described by Becker et al. (2007) (Becker, P. M. et al. J. Appl. Microbiol, 2007; 103, 2686-2696). From the time at which the bacterial growth reached an OD of 0.05 (tOD=0.05, h), the log CFU were calculated for each fraction using the described linear models for each in vitro test (Gonzalez-Ortiz, G. et al. Br. J. Nutr. 2013: 1-10). Significant differences on the log CFU among fractions were determined by one-way analysis of variance (ANOVA) by using the GLM procedure of the statistical package of SAS 9.2 (SAS Inc.; Cary, N.C., USA). Linear, quadratic and cubic contrasts were performed to analyse the dose-response of WB and the >300 kDa fraction in the blocking test. Differences between means were tested by the Tukey-Kramer adjustment for multiple comparisons.

For all analyses, the criterion for significance was P<0.05.

### The Anti-Biofilm Activity of WB Soluble Extract.

A minimal inhibitory concentration-assay was performed to discard any possible inhibitory effect of WB-extract on the S. aureus growth. It was confirmed that bacterial growth was not inhibited during the incubation overnight with WB-extract nor with the fractions obtained by molecular weight (MW).

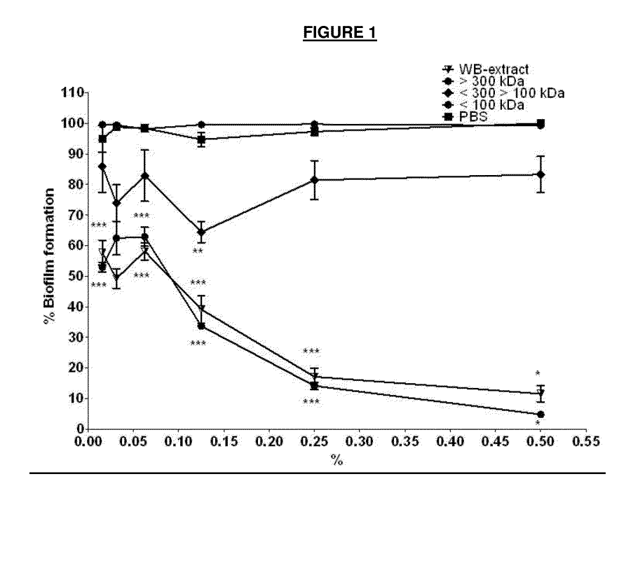

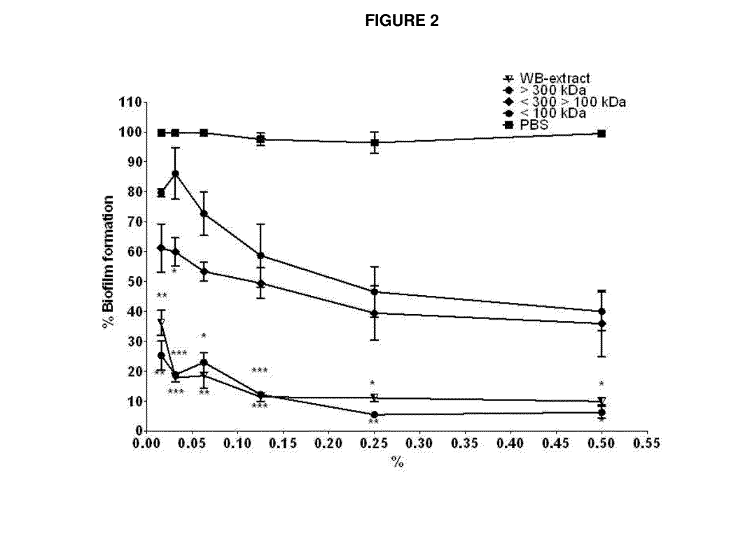

FIGS. 1 and 2 show the results of the biofilm inhibition and destruction assays for the WB-extract and its fractions at different concentrations. At the higher concentration (equivalent to a 0.5% WB suspension) the soluble extract from WB showed a clear anti-biofilm capacity, inhibiting the biofilm formation (P=0.05) and also the destruction (P=0.02) of the biofilm previously formed compared to PBS (less than 11.52%±1.62 and 9.93%±1.92 of biofilm formation, respectively). All the rest of decreasing concentrations tested were also shown to be statistically effective (P·0.05) in a dose response picture. With the lowest concentration assayed (equivalent to 0.016% WB suspension) the biofilm formed represent less than 57.75%±2.75 and 36.21%±4.18 compared to PBS (P=0.006 and P<0.0001) for the biofilm inhibition and destruction assays, respectively.

The fractionation by MW of the soluble WB-extract led to three different fractions to test, the >300-kDa, the <300>100-kDa and the <100-kDa. Showing a very similar response as the WB extract did, the >300-kDa fraction had a significant anti-biofilm activity in both in vitro assays at all concentrations tested (P<0.05) (FIGS. 1 and 2). In the biofilm inhibition assay, the half maximal inhibitory concentration (1050) for WB-extract and the >300-kDa fraction were 0.06%±0.01 and 0.07%±0.01, respectively.

On the other hand, in the biofilm destruction assay, the 10₅₀ of both samples were lower than 0.016% (FIG. 2), thus suggesting that WB extract and the >300-kDa fraction are even more active in destroying the biofilm at lower concentrations than inhibiting the biofilm formation. No significant anti-biofilm effects were found regarding to the <300>100-kDa and the <100-kDa fractions. These findings confirm that the active molecule or molecules present in WB-extract against S. aureus biofilm are inside the >300-kDa fraction.

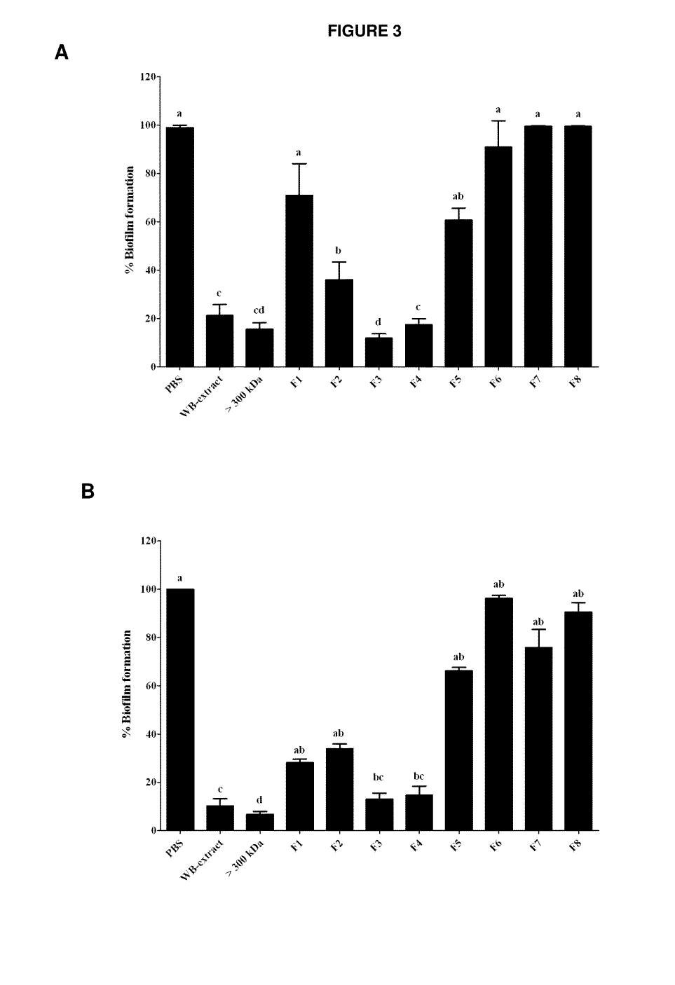

After the size exclusion chromatography of the >300 kDa fraction, eight fractions were obtained to identify where the active anti-biofilm capacity remains. F3 and F4 demonstrated to possess ability to inhibit biofilm formation (FIG. 3A) and to destroy biofilm (FIG. 3B) compared to the other fractions which none effect was detected. In both in vitro assays, these two fractions evidenced anti-biofilm properties as the WB-extract and the >300 kDa at 0.5% of concentration.

### The Interference of WB in the AHL Based QS System.

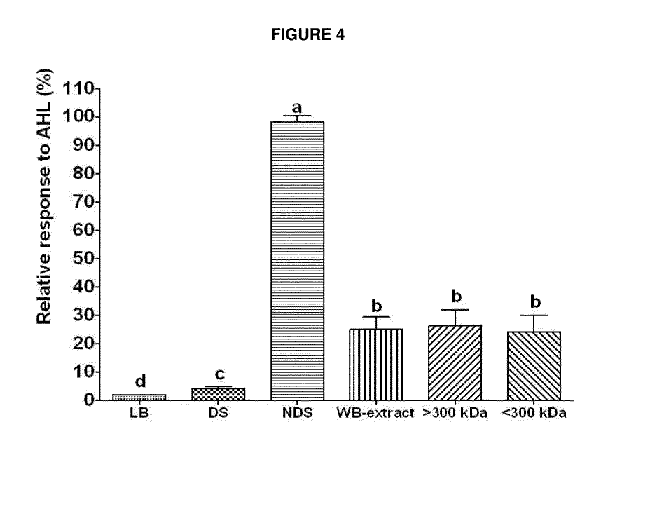

Additionally, the ability of WB extract and the fractions >300- and <300-kDa to degrade AHL was measured and therefore to interfere in QS pathways based on this bacterial signal. In FIG. 4, the relative response of AHL of each treatment compared to the positive control is illustrated. As expected, the AHL-degrader control strain (DS) presented a very low relative response, 4.3%±0.67, whereas the AHL non-degrader control strain (NDS) showed a high value of the relative response to AHL, 98.2%±2.32. The WB-extract also showed significant reductions in the AHL response with a mean value of 25.0%±3.17 and similar values were also found for both MW fractions that did not show differences between them (26.24%±4.00 and 24.26%±3.74 values for >300- and <300-kDa fractions, respectively).

Insights into the Possible Carbohydrate or Proteinaceous Nature of the Functional Molecule.

Since anti-adhesives based on carbohydrate structures are more common than those based on proteins (Wittschier, N. et al. Pharm. Pharmacol. 2007; 59, 777-786; Pieters, R. J. Adv. Exp. Med. Biol. 2011; 715, 227-240), we decided to check whether carbohydrates were involved in ETEC K88 binding. A carbohydrate digestion was carried out by using both O-glycosidase and neuraminidase in order to detect carbohydrate participation in the ETEC K88 binding. The O-glycosidase, also known as endo-•-N-acetylgalactosaminidase, catalyzes the removal of Core 1 (Gal•(1-3)GalNAc-•-O-Ser/Thr) and Core 3 (GlcNAc•(1-3)GalNAc-•-O-Ser/Thr)O-linked disaccharides from glycoproteins. Meanwhile the neuraminidase is an exoglycosidase enzyme which catalyzes the hydrolysis of •2-3, •2-6, and •2-8 linked N-acetyl-neuraminic acid residues from glycoproteins and oligosaccharides maximizing the disappearance of sugars attached to proteins. The digestion of the soluble extract of WB did not modify the adhesion ability compared to the non-incubated WB, indicating that carbohydrates do not play any important role in the recognition of ETEC K88.

Wheat bran is a by-product very rich in different components with a protein content of about 151 to 221 g/kg dry matter (Rosenfelder, P. et al. Anim. Feed Sci. Tech. 2013; 185, 107-125). Previous investigations suggested the participation of a protein or a glycoprotein in the recognition of ETEC K88. Therefore, a simple heat treatment of WB-extract was conducted to verify the implication of a protein-derived compound in the recognition of the bacteria. The heat treatment at 90° C. for 30 min reduced the number of ETEC K88 attached to wells to levels similar to PBS as compared to the WB-extract which significantly attached more bacteria (5.42 vs. 5.39 vs. 6.68 log CFU per well respectively; P<0.0001). This result is consistent with the implication of a protein from WB in the attachment of ETEC K88.

Identification of the Molecular Weight Fraction that Contributes in the Recognition of ETEC K88.

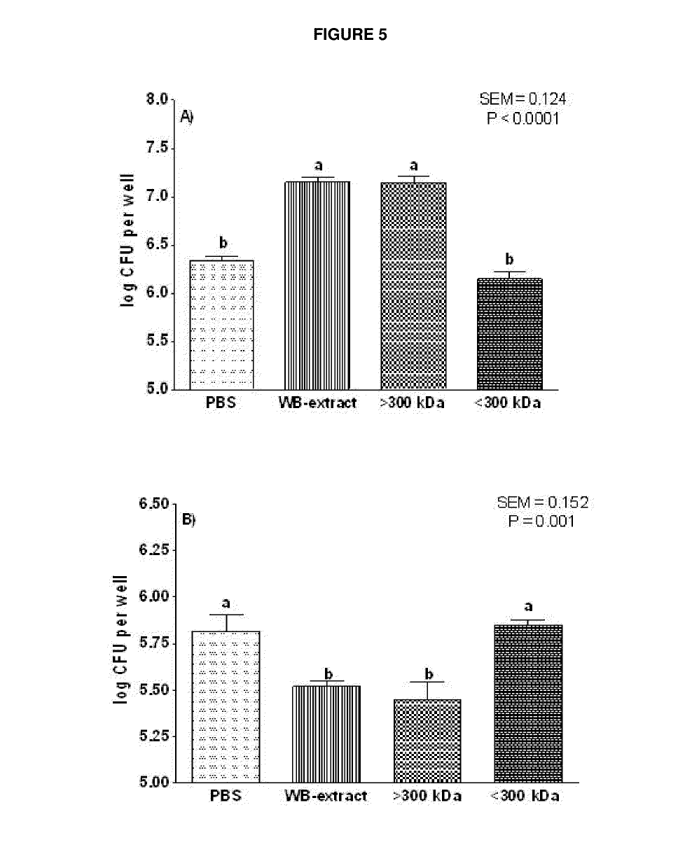

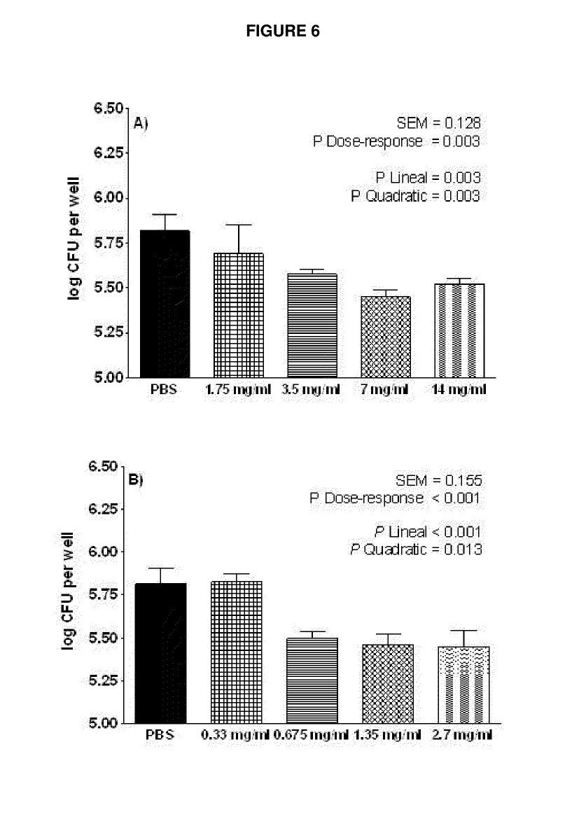

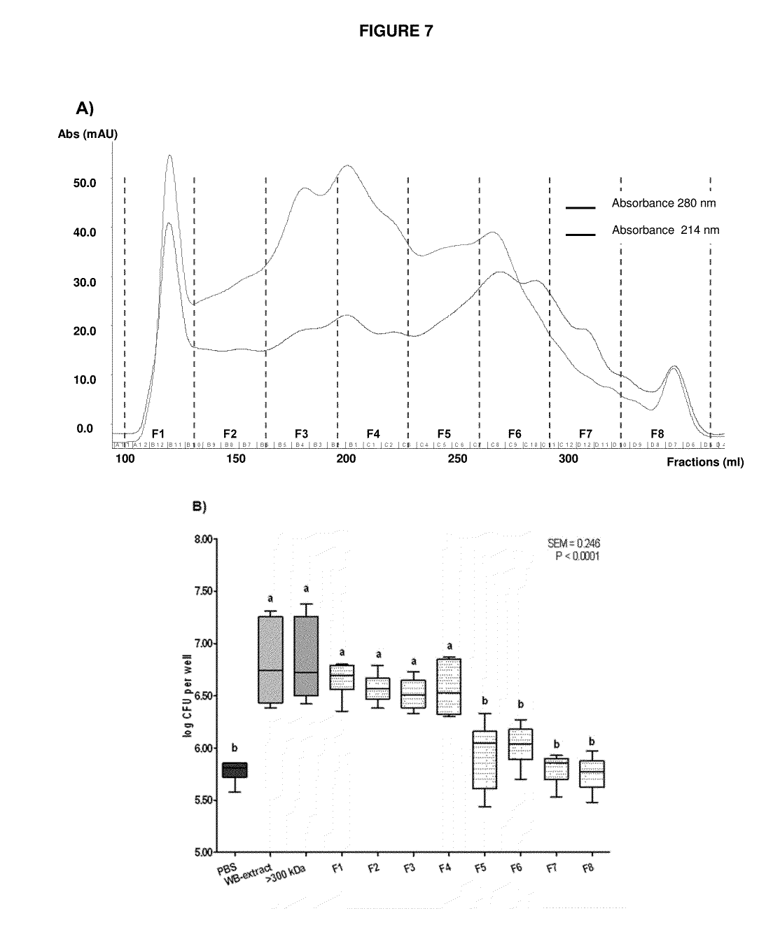

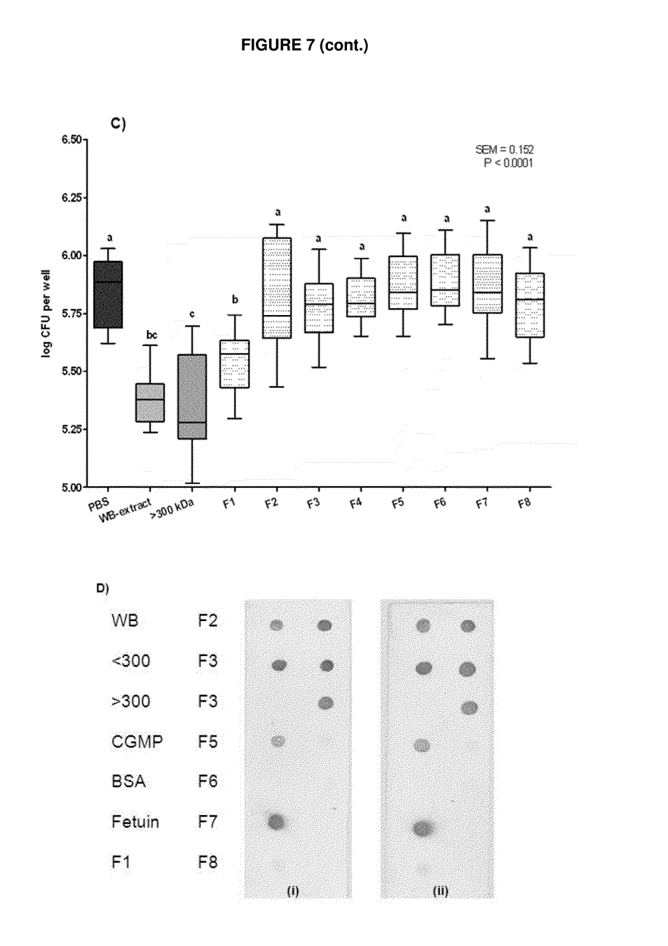

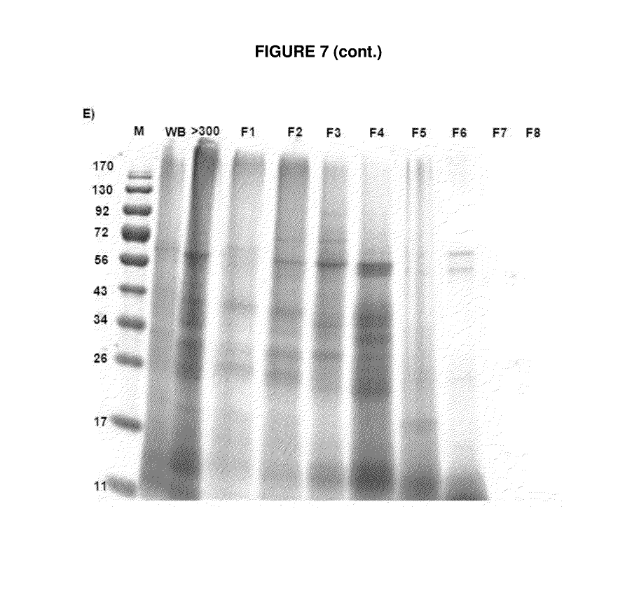

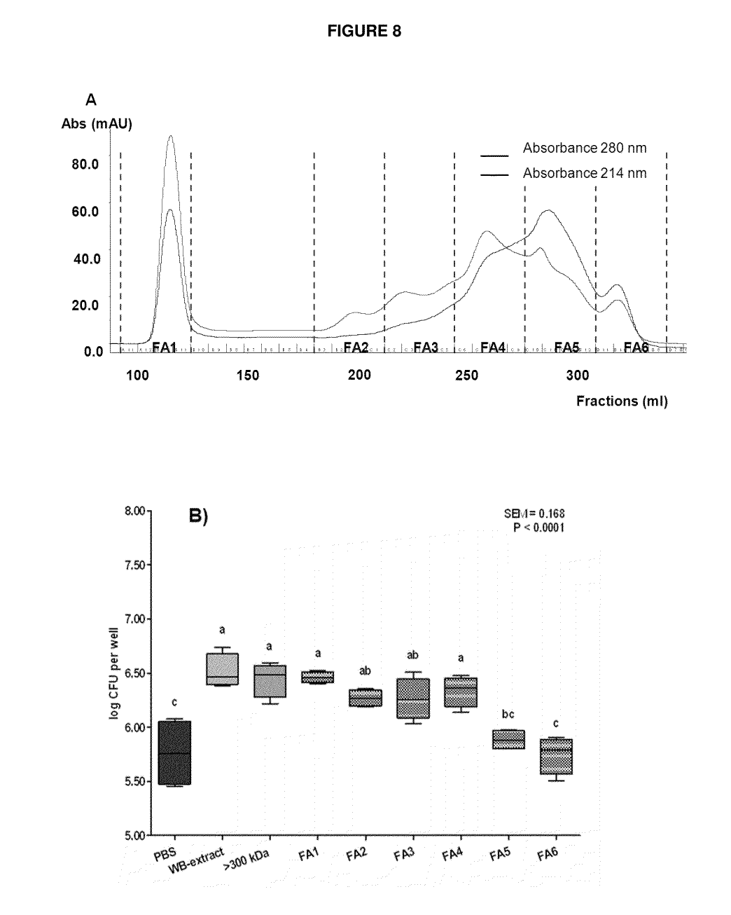

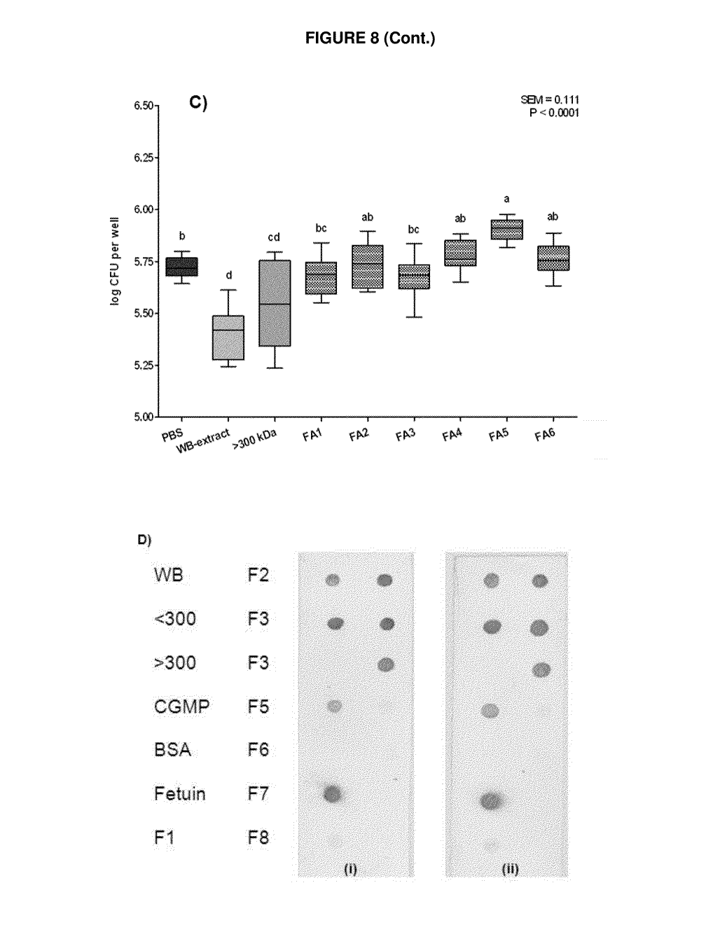

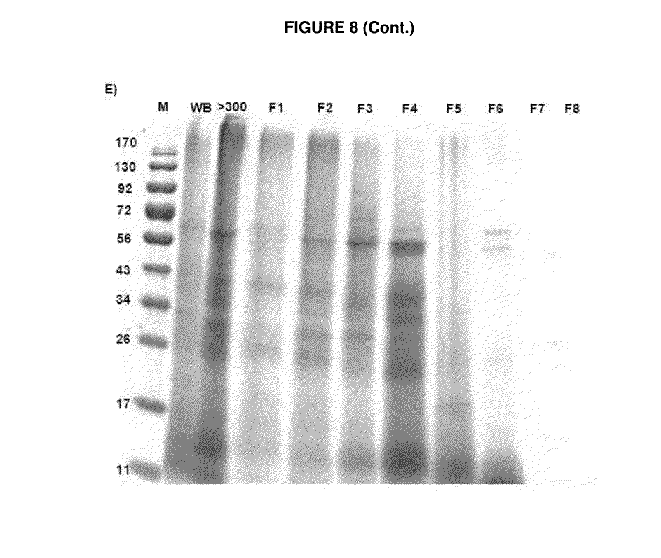

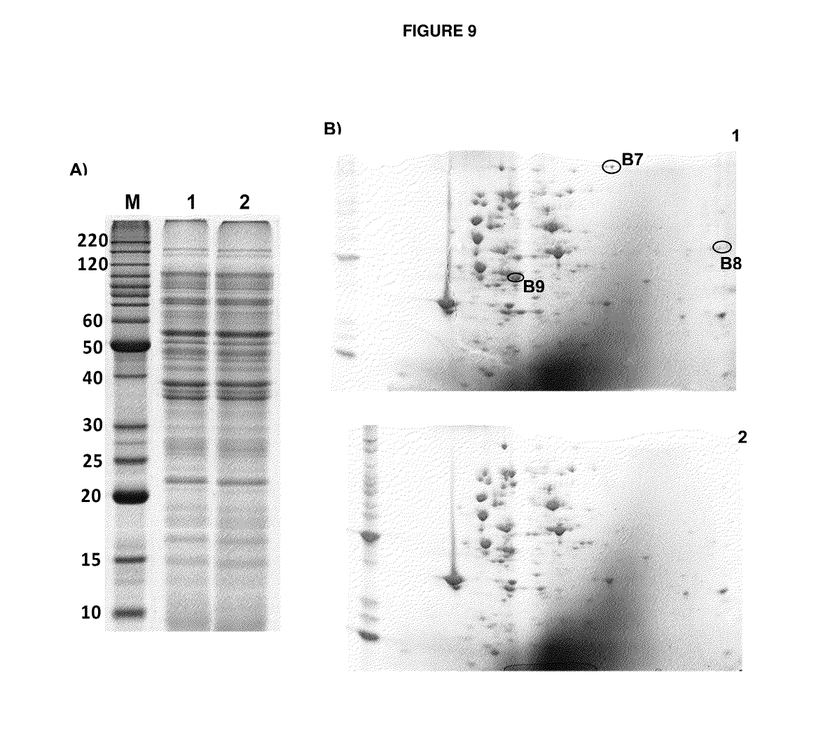

The fractionation by MW of the soluble extract of WB using a 300,000-Da cut-off size filter resulted in four different fractions: >300-kDa, <300-KDa, <300>100-kDa and <100-kDa. For the present example, it was used the fractions >300-kDa and <300-kDa. The sample concentrations included in the in vitro adhesion test and blocking test were fixed according to an equivalent of an extract at 10%. Therefore, the WB extract and the >300-kDa and <300-kDa fractions were tested at 14 mg/mL, 2.7 mg/mL and 17 mg/mL respectively. Results obtained in the adhesion test as well as in the blocking test with the different fractions (FIG. 5) revealed that the fraction adhering and blocking ETEC K88 attachment to IPEC-J2 was the >300-kDa; whereas the <300-kDa fraction did not modify the number of ETEC K88 adhered or blocked compared to PBS in both in vitro assays.

A dose-response assay was carried out with the WB extract and the >300-kDa fraction in a wide range of concentrations (FIG. 6). The linear and quadratic response were significant for different doses in the WB-extract (P<0.05) (FIG. 6A) and also in the >300-kDa fraction (FIG. 6B) demonstrating their anti-adhesive ability.

On the basis of these results, a proteinaceous compound from the >300-kDa fraction seems to mediate the recognition of ETEC K88. Therefore, a 1D SDS-PAGE was performed to compare the protein profile among the WB-extract and the >300-kDa and the <300-kDa fractions to detect a specific band that could be involved in the fimbriae recognition process. Different protein profiles were visualized in the 1D SDS-PAGE. However, almost no bands in the <300-kDa fractions were detected. The WB extract and the >300-kDa fraction shared several protein bands and many of them displayed a MW below 90-kDa. This fact is quite unexpected, since one of the fractions should mostly contain proteins with a MW higher than 300-kDa. These findings could indicate that the target protein belongs to a high MW multicomponent protein complex (>300-kDa) which is disrupted under the denaturing conditions of the SDS gel and renders the individual proteins.

### Characterization of the Active Molecule by Size Exclusion Chromatography.

Size exclusion chromatography (SEC) was performed to fractionate the >300-kDa fraction and to isolate the target protein. Eight SEC fractions (FIG. 7A) were obtained (F1 to F8) and subsequently evaluated in the in vitro adhesion test and blocking test and dot blot assay. Results obtained from the adhesion test, revealed that F1, F2, F3 and F4 had roughly the same number of ETEC K88 cells bound as displayed by the >300-kDa fraction and the WB extract (FIG. 7B). Fractions from F5 to F8 were not able to bind ETEC K88, as it was expected due to the low-molecular weight proteins that were contained in these samples. The binding of F1 to F4 fractions to ETEC K88 cells may suggest the presence of a protein in these samples that is similar to the target protein on the tissue surface that is recognized by the fimbriae. When the same fractions were evaluated in the blocking test assay, the main difference was found in the F1 fraction, which was the only one able to interfere in the ETEC K88 adhesion to IPEC-J2 by reducing the number of attached bacteria (FIG. 7C). This result shows that the chromatographic separation allowed us to isolate the protein with blocking activity of the >300-kDa sample in a single fraction, F1. Also, it indicates that there are two distinct functions in the >300-kDa sample, an adhesive property observed in fraction F1 to F4 and a blocking function located in fraction F1. In FIG. 7D, the results of dot blot assays are represented. Both K88ac fimbriae, coming from two different enterotoxigenic E. coli strains (FV12408 and 5/95), bind to WB-extract and >300-kDa fraction but not to the <300-kDa fraction. Regarding the 8 SEC fractions obtained from the >300-kDa fraction, K88ac fimbriae bind strongly to F2, F3 and F4 fractions, and less intensely to F1. Similarly to the adhesion test and blocking test assays, no binding signals were detected for F5, F6, F7 and F8 fractions. The 8 SEC fractions were further separated by SDS-PAGE gel and compared (FIG. 7E). Surprisingly, F1, F2, F3 and F4 contain several low MW proteins. The presence of these low MW proteins in a fraction separated by a filter of 300-kDa could confirm the previous hypothesis regarding the existence of protein complexes in the >300-kDa fraction, that are individualized under the denaturing conditions of the gel. Accordingly, the >300-kDa fraction was treated with acetonitrile (Panreac Química S.L.U.) and subsequently fractionated by SEC in the presence of ACN to break down the potential complexes without completely denaturing the proteins, in order to keep their activity. As a result of this chromatography (FIG. 8A), six fractions were obtained (FA1 to FA6) and evaluated by both in vitro adhesion test and blocking test and dot blot.

In the adhesion test, the SEC fractions FA1 to FA4 showed a similar effect to that of >300-kDa fraction and WB extract, binding a high log CFU of ETEC K88 (FIG. 8B) and showing that the ACN treatment did not affect their binding capacity. These results are in accordance with the dot blot results (FIG. 8D), which show that both types of K88ac fimbriae bind strongly to FA2 and FA3, and with lower intensity to FA1 and FA4. In contrast, those active fractions in the adhesion test and dot blot assay were not able to interfere in the ETEC K88 attachment to IPEC-J2 (FIG. 8C).

The 1D SDS-PAGE electrophoresis did not reveal a clear different band pattern among fractions (FIG. 8E). Six bands (B1, B2, B3, B4, B5 and B6) which were in common among the active fractions in the adhesion test and dot blot (FA2 to FA4) were excised to identify the proteins by Matrix Assisted Laser Desorption/Ionization Time-of-Flight (MALDI-TOF) mass spectrometry (MS). Table 1 sums up the most important information regarding the identification of the excised bands by MS using Mascot search engine. Bands B1 and B2, shared the same beta amylase and B1 also contained a protein disulphide isomerase 2 precursor of 56 kDa. In FA4, the high intensity of bands distributed at lower MW, allowed us to excise more bands for identification. Serpin-Z2B and Class II chitinase, were found to be the most representative proteins in B3 and B4 bands, respectively. Finally, two protease inhibitors were identified in B5 and B6 with a low MW of approximately 19 kDa.

### Isolation of Wheat Bran Proteins Attaching to ETEC K88.

In parallel, an in vitro experiment was conducted which consisted in incubating ETEC K88 cells with the WB extract, in order to rescue the WB molecules most firmly attached to the bacteria. The material retrieved after the shaving process was used to perform 1D and 2D gels with both samples and to identify the spots which were only detected in the presence of WB (FIG. 9). In the 1D gel it was not possible to identify any differential bands between the incubation of bacteria with PBS or WB extract due to the high number of proteins in the sample. The two 2D gels were compared using the Progenesis® Same Spots® software (Nonlinear Dinamics) and after the alignment of the images six spots with a fold>2 were detected. Three of the spots belonged to the same train of spots (FIG. 9b, spot number B7) and contained a bacterial protein (See Table 1), as did the spot number B9. Finally, two spots belonging to the same train of spots (FIG. 9b, spot number B8) were identified as a protein from Triticum aestivum, the globulin 3 (See Table 1).

As conclusion, the present invention shows new therapeutical effects related to WB extracts. The anti-biofilm ability of WB extract against pathogen bacteria may be related with the presence of defence compounds in the plant and/or enzymes that could inhibit different bacterial enzymatic process or catalyse synthesised biofilm components. Moreover, some compounds in the WB extract are able to interfere with the AHL activity opening the possibility of a new anti-pathogenic effect throughout an interference with the QS of bacteria. Moreover, protease inhibitors and Globulin 3 from WB extract are involved in the binding to enterotoxigenic bacteria cells.

TABLE 1
Identification of proteins present in the soluble extract of wheat bran (Triticum aestivum)
after fractionation by MALDI-TOF (Spot no from B1 to B6). Identification of proteins rescued
after incubation between ETEC K88 and WB extract (Spot numbers from B7 to B9).

| | | | | | | | |
|---|---|---|---|---|---|---|---|
| | | | | | Protein | | Seq. |
| Spot | Accession | Protein | | Mascot | MW | Pep | Cov. |
| No. | number | description | Taxonomy | Score | (Da) | No.^{a} | (%)^{b} |

Proteins present in the soluble extract of wheat bran (Triticum aestivum) after fractionation

| | | | | | | | |
|---|---|---|---|---|---|---|---|
| B1 | gi\|474451266 | Beta amylase | Triticum urartu | 93 | 58,710 | 14 | 29 |
| | gi\|13925726 | Protein disulfide isomerase | Triticum aestivum | 70 | 56,406 | 8 | 24 |
| | | 2 precursor | | | | | |
| B2 | gi\|474451266 | Beta amylase | Triticum urartu | 131 | 58,710 | 14 | 28 |
| B3 | gi\|473793747 | Serpin-Z2B | Trititicum urartu | 79 | 45,112 | 12 | 36 |
| B4 | gi\|62465514 | Class II chitinase | Triticum aestivum | 64 | 28,200 | 13 | 87 |
| B5 | gi\|123975 | Endogenous alpha- | Triticum aestivum | 313 | 19,621 | 21 | 92 |
| | | amylase/subtilisin inhibitor | | | | | |
| B6 | gi\|123957 | Alpha-amylase/trypsin | Triticum aestivum | 85 | 18,209 | 6 | 57 |
| | | inhibitor CM3 | | | | | |

Proteins rescued after incubation between ETEC K88 and WB-extract

| | | | | | | | |
|---|---|---|---|---|---|---|---|
| B7 | gi\|260870699 | RNA polymerase, beta | Escherichia coli | 286 | 155,048 | 32 | 26 |
| | | subunit | | | | | |
| B8 | gi\|215398470 | Globulin 3 | Triticum aestivum | 122 | 66,310 | 21 | 41 |
| B9 | gi\|300946929 | Translation elongation | Escherichia coli | 342 | 44,822 | 41 | 94 |
| | | factor Tu | | | | | |

^{a}Represent the number of identified peptides by PMF.
^{b}Represent the percentage of identified peptide coverage in total sequence of protein

1. A wheat bran soluble extract for use as bacterial anti-biofilm agent.

2. The wheat bran soluble extract for use according to claim 1 as bacterial anti-adhesive agent.

3. The fraction of wheat bran soluble extract for use according to claim 1.

4. The wheat bran soluble extract for use according to claim 3, wherein the wheat bran soluble extract comprises at least one protease inhibitor selected from: Serpin-Z2B, Class II chitinase, endogenous alpha-amylase/subtilisin inhibitor and alpha-amylase/trypsin inhibitor CM3.

5. The wheat bran soluble extract for use according to claim 3 wherein the wheat bran soluble extract comprises Globulin 3 of wheat.

6. The wheat bran soluble extract for use according to claim 1, wherein the biofilm forming bacteria is from genus Staphylococcus and Escherichia.

7. The wheat bran soluble extract for use according to claim 6, wherein the bacteria are from the species S. aureus and/or E. coli.

8. The wheat bran soluble extract for use according to claim 1 in the prevention and/or treatment of diseases induced by biofilm-forming bacteria.

9. The wheat bran soluble extract for use according to claim 1, in the prevention and/or treatment of intestinal diseases.

10. A protease inhibitor selected from Serpin-Z2B, Class II chitinase, endogenous alpha-amylase/subtilisin inhibitor and alpha-amylase/trypsin inhibitor CM3 and/or Globulin 3 of wheat for use as bacterial anti-biofilm agents and/or bacterial anti-adhesive agents.

11. The protease inhibitors and/or Globulin 3 of wheat for use according to claim 10 in the prevention and/or treatment of diseases caused by pathogenic biofilm-forming bacteria.

12. A wheat bran soluble extract to treat mastitis caused by biofilm forming bacteria.

13. A fraction >300-Kd of the wheat bran soluble extract to treat mastitis caused by biofilm forming bacteria.

14. A protease inhibitor selected from Serpin-Z2B, Class II chitinase, endogenous alpha-amylase/subtilisin inhibitor and alpha-amylase/trypsin inhibitor CM3 and/or Globulin 3 of wheat bran soluble extract to treat mastitis caused by biofilm forming bacteria.

15. A protease inhibitors and/or Globulin 3 of wheat bran soluble extract to treat mastitis caused by biofilm forming bacteria.

16. The pharmaceutical, prebiotic or functional food composition comprising at least one of the following constituents:
wheat bran soluble extract as defined in claim 1, or
a protease inhibitor selected from Serpin-Z2B, Class II chitinase, endogenous alpha-amylase/subtilisin inhibitor and alpha-amylase/trypsin inhibitor CM3, or globulin 3 of wheat.

17. The composition for bacterial control of facilities or devices selected from: industrial facilities, laboratories, including branches, farms, facilities, medical devices, water of liquid feed distribution system in farms, food industry, dairy industry, pipe networks for fluids, including air-conditioning systems comprising at least one of the following constituents:
wheat bran soluble extract as defined in claim 1, or
a protease inhibitor selected from Serpin-Z2B, Class II chitinase, endogenous alpha-amylase/subtilisin inhibitor and alpha-amylase/trypsin inhibitor CM3; or globulin 3 of wheat.
